(19) United States
(12) Patent Application Publication
(10) Pub. No.: US 2023/0023897 A1
(43) Pub. Date: Jan. 26, 2023

(54) SYSTEM AND METHOD FOR CELL CHARACTERISATION IN BIOLOGICAL TISSUE

(71) Applicants: The University Court of the University of Glasgow, Glasgow (GB); THE UNIVERSITY COURT OF THE UNIVERSITY OF EDINBURGH, Edinburgh (GB)

(72) Inventors: John Richard MERCER, Glasgow (GB); Steven NEALE, Glasgow (GB); Anubhav BUSSOOA, Glasgow (GB); Daniel HOARE, Glasgow (GB); Nosrat MIRZAI, Glasgow (GB); Jakub CZYZEWSKI, Glasgow (GB); Michael HOLSGROVE, Glasgow (GB); Srinjoy MITRA, Edinburgh (GB); Andreas TSIAMIS, Edinburgh (GB); Jamie MARLAND, Edinburgh (GB)

(73) Assignees: The University Court of the University of Glasgow, Glasgow (GB); THE UNIVERSITY COURT OF THE UNIVERSITY OF EDINBURGH, Edinburgh (GB)

(21) Appl. No.: 17/443,410

(22) Filed: Jul. 26, 2021

(51) Int. Cl. A61B 5/0537; A61B 5/00; A61B 5/0538; A61N 1/375

(52) U.S. Cl. CPC A61B 5/0537; A61B 5/0002; A61B 5/6862; A61B 5/0538; A61B 5/4836; A61N 1/37516; A61B 2560/04

(57) ABSTRACT

A system that provides the ability to integrate both diagnostic tissue detection and therapeutic tissue treatment functionality in an implantable medical device (IMD), such as a graft or stent. The system comprises: an IMD having a pair of electrodes configured to contact biological tissue at an implant location; an impedance sensor configured to detect data indicative of a complex impedance of the biological tissue at the electrodes for a plurality of frequencies; and an analysis device configured to determine one or more cell types in the biological tissue using information indicative of variation in phase and magnitude of the complex impedance across the plurality of frequencies. The system may include a signal generator for applying a treatment signal to the same electrodes.

### FIELD OF THE INVENTION

The present invention relates to a system and method for cell characterisation and treatment in biological tissue.

### BACKGROUND

Cardiovascular disease (CVD) is the largest cause of human mortality in the world, with 17.5 million deaths per annum according to the World Health Organisation (WHO) and a cost of €210 billion per annum according to the European Heart Network. Two thirds of these deaths are caused by atherosclerosis, which is the furring up or blocking of arteries with cellular and fatty deposits termed “plaques”.

Blocked arteries often go undetected. Atherosclerotic plaque formation is ubiquitous across many blood vessels but is particularly dangerous within the fine coronary arteries of the heart and the cerebral vasculature of the brain. These deposits drive an inflammatory response that impinges on blood flow, depriving downstream tissues of oxygen. Over time, plaques can harden through calcification and are prone to rupture. When the plaque ruptures, clot enters the circulation, which can lead to a stroke or myocardial infarction (MI), also known as a heart attack.

Current clinical treatments for cardiovascular disease rely on lifestyle modifications and/or medications such as blood pressure, lipid lowering drugs and anti-thrombotic drugs such as clopidogrel to thin the blood. Surgical intervention is limited to coronary artery bypass grafting (CABG) or stenting of the affected vessel via percutaneous coronary intervention (PCI).

CABG is a highly invasive, time consuming and expensive operation. The patient is anesthetised, and their chest opened to allow the surgeon access to the heart and an explanted peripheral vessel is then used to bypass the occlusion caused by the plaque.

PCI is a less invasive procedure in which a stent is expanded to reopen the vessel. PCI is well tolerated and is associated with faster recoveries times than CABG. PCI's drawback is that stents damage the lining of the vessel, causing a wound response that drives re-occlusion, which is called “in-stent restenosis” (ISR). ISR is caused because when the stent is deployed, the vessel wall is denuded of the endothelial layer, thereby leading to the migration and hyperplasia of smooth muscle cells that can cause re-blockage of the artery.

ISR is a significant challenge as it often goes undetected and, despite stent improvements, ISR remains difficult to avoid.

In an attempt to reduce the risks of ISR, drug eluting stents (DES) have been developed, which include coatings of anti-proliferative drugs (such as Rapamycin (Sirolimus)), which can be classified as either cytostatic or cytotoxic drugs. The polymer used to load the drugs is associated with local inflammatory reactions and will non-specifically elute off the stent. However, in up to 10% of cases, restenosis will still form and be completely undetected.

Additionally, a Cochrane systematic review found that DESs only slightly reduced the absolute risk of a major adverse cardiovascular event (MACE) compared to bare metal stents (BMS 6.63% 19 versus DES 6.36%. Indeed, the most recent generation of DESs have a significantly lower patient readmittance rate for target lesion revascularization, (2.91% versus 12.32%), but compared to the first generation DESs have only a modest improvement of 2.91% versus 4.34%.

Improvements in stent design such as reduction in stent strut thickness have also driven improvements. New technologies in bioactive polymeric drug release systems that control coating thickness, surface roughness, drug load and drug elution kinetics are also making significant improvements. However, these are still hindered by protracted antiplatelet regimes used to prevent clot formation which are not appropriate for patients with multiple co-morbidities.

With over 100,000 PCI procedures performed in the UK every year and with >3 million deployed globally, many thousands of patients will suffer complications from their stent.

Increasingly, synthetic grafts (rather than stents) are being used to bypass disease segments of vessels. However, wherever a synthetic and native vessel join, a similar wound response occurs that can also silently re-block the vessel.

In view of the above, it would be desirable to provide an “early warning system” for implantable medical devices that can detect the very earliest changes associated with vessel occlusion and which could stratify patient need.

To date, micro-electro-mechanical systems (MEMs) using inductance based capacitor systems capable of pressure measurement have been explored when combined with a stent. While other research has centred around resonator pressure sensors, and diaphragm based pressure sensors for stents. Although relatively sensitive to detecting a pressure and flow change within a target vessel, they are bulky and require a severe blockage (>70%) to be present in order to detect the blockage. Thus, they are only able detect late clinical events. In contrast, the ability to “sense” ISR early before flow disruption has occurred would offer a significant clinical advantage.

### SUMMARY OF THE INVENTION

At its most general, the invention relates to a system that provides the ability to integrate both diagnostic tissue detection and therapeutic tissue treatment functionality in an implantable medical device (IMD), such as a graft or stent.

According to a first aspect of the invention, there is provided a system for cell characterisation in biological tissue, the system comprising: an implantable medical device, IMD, comprising a plurality of sensing elements configured to interact with biological tissue at an implant location; an impedance sensor configured to: apply a sensing AC signal to the plurality of sensing elements, vary a frequency of the AC signal across a detection range; and detect data indicative of a complex impedance of the biological tissue at the plurality of sensing elements for a plurality of frequencies in the detection range; and an analysis device configured to: receive the detected data from the impedance sensor; and determine one or more cell types in the biological tissue using information indicative of variation in phase and magnitude of the complex impedance across the plurality of frequencies.

The system therefore provides an in vivo determination of cell type, thereby providing a remote monitoring system that enables the early detection and reporting of cell changes within the body.

For example, this may be employed in an IMD to detect the presence of smooth muscle cells and/or endothelial cells and thereby assess even the earliest changes associated with ISR. Based on the cell type detected, the degree of healing at the IMD may be assessed, and potential blockages caused by blood clots or other issues may be identified even at an early stage. Appropriate treatment may then be carried out an early stage.

Although examples are provided in the context of evaluating vascular cell types at a stent/graft, the system can also be usefully employed for evaluating other cell types at other types of devices. For example, the system could be used to distinguish between cancer cells and non-cancer cells or to monitor differentiation of stem cells towards different phenotypes.

The inventors have observed that by utilising information indicative of variation in both phase and magnitude of complex impedance, across a plurality of frequencies (also referred to as a “frequency sweep”), cell type can be more reliably determined. This is because this variation is distinctive for a particular cell type, and can thus be readily distinguished from the variations for different cell types. Accordingly, a given cell type may be considered to have a particular “signature” for the variation of phase and magnitude across a plurality of frequencies, which may be used to determine cell type.

The impedance sensor may form part of the IMD. This provides an integrated arrangement that is fully implantable within the body. For example, where the IMD is a graft, the plurality of sensing elements and impedance sensor may be mounted on inner walls of the graft.

The analysis device may be configured to compare the detected data to stored signature data representative of one or more known cell types. The stored signature data may be in a memory of the analysis device, or may be stored on a remote computing device (e.g. cloud-based server) that is communicably coupled to the analysis device. In this latter example, the analysis device may be configured to communicate the detected data to the remote computing device, which performs the comparison and returns a result to the analysis device.

The stored signature data may include complex impedance spectra data indicative of a variation in phase and magnitude of complex impedance across the plurality of frequencies for each known cell type. The complex impedance spectra data may comprise information indicative of an evolution over time of complex impedance properties for each or mixed known cell types. Evaluating the evolution of complex impedance properties over time (e.g. using the rate of change over time) can usefully provide further insight into the progression of cell changes. For example, in the context of ISR, this can allow for the assessment of an amount of ISR, a rate of occlusion and a stability of the lesion. The stored signature data may include a peak-to-peak difference in phase angle change for each known cell type.

The analysis device may be configured to determine one or more cell types in the biological tissue using both phase and magnitude information in the detected data. For example, the analysis device may use a signature spectrum for each of the phase and magnitude. The signature spectrum represents change in phase or magnitude with frequency, and may provide a convenient arrangement for analysing the distinctive variations in phase and magnitude information across the variety of frequencies. The inventors have found that variations in magnitude against phase are easily assessed, since there are distinctive differences in the appearances of the plots for different cell types. For example, the cell type may be determined based on a trend in the shape of the spectrum or based on a “bulge” at a particular location along the curve of the spectrum, to provide a visually obvious method of distinguishing different cell populations. The way in which the phase angle changes between the peaks for each cell type is another way of distinguishing different cell types.

The stored signature data mentioned above may include a library of one or more signature spectra, wherein each signature spectrum within the library is representative of a different cell type. This utilises the distinctive variations in phase and magnitude information to determine a large variety of cell types. As mentioned above, the signature spectra may be stored locally, e.g. within a storage unit of the analysis device, or remotely e.g. in a server in communication with the analysis device.

The stored signature data may include data for a smooth muscle cell type and an endothelial cell type. This enables ISR to be assessed, since this is reflected by an increase in smooth muscle cells (SMCs) compared to endothelial cells (ECs). Therefore, this arrangement is particularly advantageous for use in stents and/or grafts for placement in the vascular system.

In addition to cell characterisation, the system may be used to cause apoptosis or electroporation of cells. For example, the system may include a signal generator configured to apply a treatment signal to the plurality of sensing elements. The signal generator may be formed with or within the IMD, e.g. as an additional module to the impedance sensor. The IMD can be thus provide a dual functionality, by being capable of operating in either a diagnostic (sensing) mode or a therapeutic mode. When operating in the therapeutic mode, the treatment signal is delivered to the plurality of sensing elements. The treatment signal may be an AC or DC signal. In order to provide a therapeutic effect, e.g. to kill unwanted cells, the treatment signal is arranged to deliver more energy into biological tissue at the implant location than the sensing signal. For example, the treatment signal may have a higher current component (DC or root mean squared magnitude) than the sensing signal. The system may thus provide a combined diagnostic and therapeutic biosensor, which may be transformational for the treatment of vascular diseases such as atherosclerosis and central line access.

As discussed above, the sensing and/or therapeutic functionality may be integrated in the IMD. The IMD may thus comprise a housing that contains the impedance sensor, the signal generator, and a controller arranged to configure the system to operate in either a sensing mode or a treatment mode.

The system may further comprise a communication module configured for bi-directional communication with the analysis device. The communication module may be also be contained in the housing. Two-way communication is advantageous because it allows for secure transmission of sensed data as well as receipt of instructions, e.g. for execution by the controller. For example, the controller may be configured to receive therapeutic care plans from physicians, changes in reading parameters, or to receive a request for a data reading on demand.

The communication module may also permit remote selection of the operation mode of the IMD, e.g. selecting either the sensing mode or the treatment mode. The selection may be determined at the analysis device, either manually according to a user input, or in an automated manner. For example, the analysis device may be configured to selection the treatment mode if it is determined that one or more unwanted cell types exist in the implant location. The automated selection by the analysis device may operate under a stored treatment plan for responding to different cell types, e.g. smooth muscle vs endothelial. This may be advantageous because it allows treatment to proceed with requiring user intervention.

When operating manually, the analysis device may issue an operation command based on a user input (e.g. from a physician) into the analysis device. The user may therefore control the response to a given tissue type. The operation command may select the treatment mode (and possible set parameters for the treatment signal), or may select the sensing mode e.g. by issuing a data acquisition command. In this way, the system is able to provide data on demand, when requested by a user.

Upon receipt of a data acquisition command, the impedance sensor may detect the data indicative of complex impedance by applying the sensing AC signal and varying the frequency of the AC signal upon receipt of the data acquisition command, to provide up-to-date data on request. Alternatively or in combination, the impedance sensor may also send previously detected data on receipt of the command, e.g. as a log of recent data at various points in time.

Alternatively or in combination, the impedance sensor may transmit data autonomously, without receiving a data acquisition command, e.g. by transmitting detected data based on a predetermined schedule or when the data meets a predetermined threshold.

The plurality of sensing elements may comprise a pair of electrodes. In one example, the impedance sensor may comprises a plurality of electrode pairs, where each pair of electrodes is selectively operable in the sensing mode or treatment mode. Such an arrangement may permit the location in which cell characterisation is performed to be selectable.

The pair of electrodes may be a pair of interdigitated comb electrodes. The pair of electrodes may be configured to conform to a surface of the IMD. For example, where the IMD is a graft or stent, the pair of electrodes may be arranged circumferentially to lie on or in an inner surface of the graft or stent, e.g. in a region where the graft or stent interfaces directly or indirectly with a biological tissue. The examples discussed below demonstrate that the interdigitated electrode configuration provides an environment suitable for both direct contact or contactless method for detecting impedance capable of cell characterisation and delivering energy to cause apoptosis. Moreover, it is demonstrated that this type of configuration is not only scalable in a manner that allows it to be miniaturized for use in IMDs, but that such miniaturization can actually achieve an increase in detection sensitivity.

The pair of electrodes may be formed using any suitable microfabrication technique. For example, they may be formed from conductive material (e.g. platinum, polyimide, PEDOT:PSS or other biocompatible material) applied to a silicon or non-silicon substrate.

As mentioned above, the IMD may comprise multiple pairs of electrodes configured to contact the biological tissue at multiple regions of the implant location. Such a configuration may enable cell types to be determined on both an inner layer and an outer layers of a graft or stent, or may be used to better characterise cell type in certain locations. Having multiple pairs of electrodes may also permit the treatment signal to be target, e.g. so that cell death occurs only in specific regions.

As discussed above, the IMD may be an intravascular device. For example, the IMD may be a stent, graft, or a stent/graft hybrid for placement in the vascular system. In variant embodiments, the IMD may be a different type of device, e.g. an implant for monitoring cellular changes at a tumour.

The analysis device may be intravascular or extravascular, for example a remote device in wireless communication with the impedance sensor. The remote device may relay data and power signals to a wearable device, such a smartwatch or the like. Alternatively, it may be a smartphone, tablet, laptop or other personal computer. The analysis device may be in networked communication with a cloud-based central computer that is configured to perform the comparison and determination functions discussed above. The analysis device may thus act as a relay to the cloud-based central computer and as user interface for the system e.g. to display results and/or accept user input.

In another aspect, the invention may provide a method of characterising cells in biological tissue, the method comprising: applying, by an impedance sensor, a sensing AC signal to a pair of electrodes in an implantable medical device, IMD, that are in contact with biological tissue at an implant location, varying a frequency of the sensing AC signal across a detection range; detecting, by the impedance sensor, data indicative of a complex impedance of the biological tissue at the electrodes for a plurality of frequencies in the detection range; and determining, by an analysis device in communication with the impedance sensor, one or more cell types in the biological tissue using information indicative of variation in phase and magnitude of the complex impedance across the plurality of frequencies.

Features of the first aspect may be equally applicable to the second aspect. In other words, the invention includes the combination of the aspects and preferred features described except where such a combination is clearly impermissible or expressly avoided.

### Summary of the Figures

Embodiments and experiments illustrating the principles of the invention will now be discussed with reference to the accompanying figures in which:

FIG. 1 is a schematic diagram of an experimental setup used to obtain the results discussed herein.

FIG. 2 is a schematic diagram of a system that is an embodiment of the invention.

FIG. 3 is a multiple image alignment (MIA) micrograph of large interdigitated electrode and small interdigitated electrode, respectively showing effective electrode area.

FIG. 4 is a graph showing mean increases in impedance at different frequencies due to 200000 MASMCs detected by large interdigitated electrode and small interdigitated electrode.

FIG. 5A and FIG. 5B are graphs showing detection of cell adherence over time and induction of cell death by comparison of impedance measurements of large interdigitated electrode and small interdigitated electrode performed over 45 h with a pulse voltage for 2 min applied at 20 h to induce electroporation.

FIG. 6 is a graph showing impedance measurement for the whole experiment, including during post-seeding, cell adherence, post-staining, and post-voltage application.

FIGS. 7A and 7B are graphs showing mean percentages of live, apoptotic, and necrotic cells at the different time points for (i) negative control without voltage, (ii) treatment with voltage, (iii) positive control with scratch, and (iv) positive control with 50×10⁻⁶ m etoposide.

FIG. 8 is a compiled Bode plot for MASMC, sEND1 and co-culture, acquired using a large interdigitated electrode.

FIG. 9 is a compiled Bode plot for MASMC, sEND1 and co-culture, acquired using a small interdigitated electrode.

FIG. 10 is a set of graphs showing impedance values across frequency spectrum at given time points.

FIG. 11 is a set of graphs showing phase change across frequency spectrum at given time points.

FIG. 12 is a graph showing change in area under the curve over time of impedance.

FIG. 13 is a graph showing absolute change of phase angle over time.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

FIG. 2 is a schematic diagram of a system in which a sensor 200 is integrated with an implanted medical device (IMD) such as a stent or graft for detecting and reporting ISR. The sensor may use a detection technique (described in more detail below) that advantageously permits the detection of ISR before it is clinically apparent. In particular, the sensor is configured to use electric impedance spectroscopy (EIS) as the means to characterise and distinguish different cell types associated with ISR. The experiments discussed below demonstrate that interdigitated electrodes can be used to discriminate between the very earliest changes associated with restenosis. This detection process also has the potential to detect other vascular parameters such as blood clot formation as well as vascular healing and when combined with two-way communication protocols, the system can be used both as a diagnostic and therapeutic tool.

The lower part of FIG. 2 shows one example context in which the system of the invention can used. The IMD is deployed on a synthetic graft inside the distal vein or artery (circled) to bypass a failed segment of vessel. The upper part of FIG. 2 shows a schematic diagram of a sensor 200 that can be integrated into the IMD and in wireless communication with an external device 214, which may be a wearable device (e.g. smartwatch), smartphone, tablet or the like. The external device 214 may be in network communication with further devices, e.g. via the internet or a local area network. The external device 214 may thus be used to report cellularity, pressure, and flow (detected directly or interpolated) to one or more third parties (e.g. a treating clinician or the like).

The sensor 200 comprises a housing 202 connected to a pair sensing elements, which in this example comprise a pair of interdigitated comb electrodes 204, which are configured in a substantially cylindrical shape to conform to an inner surface of the graft/vessel interface. The housing 202 comprises a controller 208, which includes a microprocessor configured to control operation of the sensor 200 according to computer readable instructions stored on a memory (not shown). The controller 208 is operable to effect three primary functions: (1) impedance sensing, (2) therapeutic treatment, and (3) two-way communication (e.g. to reporting sensed information and/or to receive information about required treatment). The housing 202 thus includes a signal generator 206 that operates to apply an AC voltage signal to the electrodes 204. When operating in a diagnostic mode, the controller 208 is configured to sweep a frequency of the voltage signal and detect complex impedance at the electrodes. Complex impedance is a vector quantity. Herein, unless the context indicates otherwise, we use the term “impedance” to refer to the magnitude of the complex impedance and the term “phase” to refer to its direction.

Based on the detected results, the controller 208 may be configured to generate spectra that represent the variation in both the magnitude and phase of impedance with frequency. The type of tissue growing on the electrodes can be determined by monitoring changes in these spectra over time. This determination may be done remotely or locally. For example, the spectra may be communicated to an external unit for analysis, or the controller 208 may be configured to analyse them in situ.

To achieve communication, the housing 202 includes a communication unit 210 and antenna 212 configured to communicate with the external device 214 using any appropriate wireless protocol. A short range protocol such as Bluetooth® may be used.

There now follows a detailed explanation of various experiments that demonstrate the functionality of the system set out above.

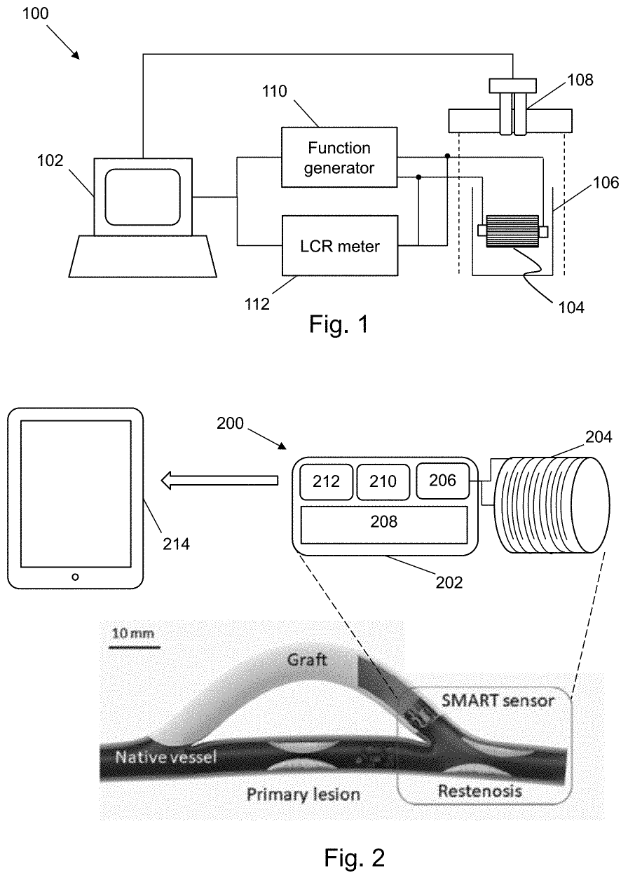

FIG. 1 is a schematic diagram that illustrate a setup 100 used in the experiments. The setup 100 comprises a computer 102 that performs the operations of the controller discussed above. The computer 102 is connected to control a function generator (AimTTi-TG5011A, UK) 110 and an LCR meter (Hioki IM 3536, Japan) 112, which are in turn connected to a pair of interdigitated electrodes 104.

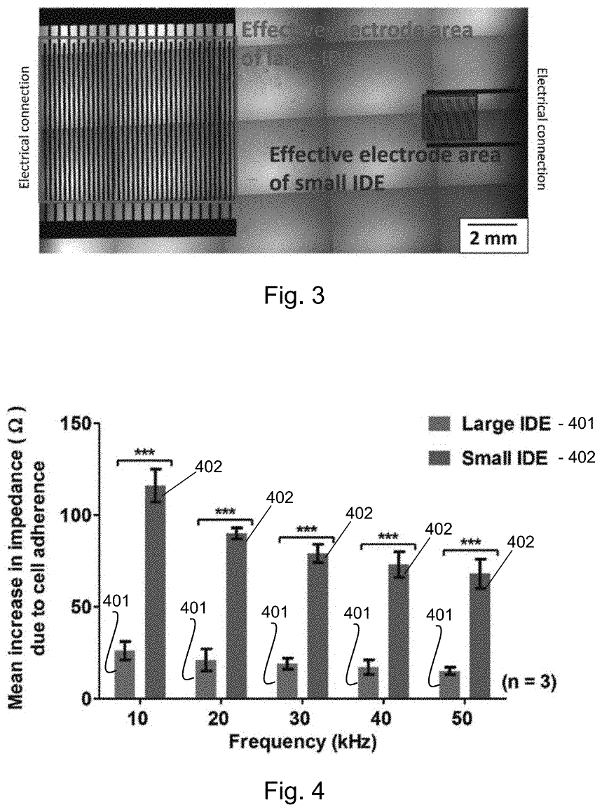

In the following experiments, two types of interdigitated electrodes (IDEs) were used. FIG. 3 is a photograph of the interdigitated electrodes used in the experiments. The IDEs were fabricated at the James Watt Nanofabrication Centre of University of Glasgow using standard microfabrication techniques. The first type of IDE is a large IDE (4mm2) consisting of two sets of 20 fingers (length 800 μm, width 100 μm, and 100 μm separation between each finger). The second type of IDE is a small IDE (0.5 mm²) consisting of two sets of 20 fingers (length 200 μm, width 25 μm, and 25 μm separation between each finger). Each set of fingers in the IDE 104 has a corresponding contact pad. The IDE is connected to the function generator 110 and LCR meter 112 via electrical wires that are soldered to the contact pads.

In one set of experiments, the electrodes were patterned on a glass microscope slide with a 10 nm titanium adhesion layer and 100 nm gold layer. However, it is also possible to fabricate the electrodes using other standard microfabrication techniques, e.g. using platinum on a silicon substrate.

In the experiments, it was also desirable to observe the surface of the IDE. Accordingly, the IDE were mounted in a temperature and CO₂ controlled enclosure 106, where they were observable via a live cell microscope 108 (Olympus IX71, Japan), that was in communication with the computer 102.

In use, the IDEs 104 were connected to the LCR meter 112 for impedance measurements and to the function generator 110 for applying controlled voltages to achieve therapeutic effects (e.g. electroporation).

The fabricated device was sterilized by misting with 70% ethanol and washing with deionized water. Baseline impedances were first measured with a culture medium only. The LCR meter 112 was configured in a fixed current (10 μA), varying voltage mode to measure impedance. The impedances were measured at 10, 20, 30, 40, and 50 kHz for both the large IDE and small IDE.

An adherent primary vascular cell type, mouse aortic smooth muscle cells (MASMCs), was used for a first batch of experiments. The cells were cultured in phenol red free DMEM media supplemented with 10% fetal bovine serum (FBS) in cell culture flasks. Once sub-confluent, the cells were trypsinized and 200,000 MASMCs were then seeded into the fabricated device, and the cell suspension was pipetted a few times to ensure a homogeneous population was plated. MASMCs were spherical when in suspension and spindle-shaped, once adhered to the culture substrate. The cells were allowed to settle down for 18 h inside an incubator (37° C. and 5% CO₂) to ensure that they adhered to the electrodes. Experimental impedances were then recorded. The increases in impedance were calculated at each frequency for the large and small IDEs. Bright field imaging and impedance measurements (at 10 kHz) were simultaneously started and acquired at 15 min intervals. At the 20 h time point, the LCR meter 112 was disconnected from the electrodes, and the function generator 110 was connected so as to apply a treatment signal, which in this example was a sine voltage of 2 Vₚₑₐₖ₋ₜₒ₋ₚₑₐₖ 40 kHz for 2 min, the minimum duration required to observe a drop in impedance.

The impedance readings obtained in the first period were thus independent of the applied voltage from the function generator. In contrast, changes in impedance after the treatment signal was applied were directly due to changes in the cellularity across the sensor surface.

Following application of the treatment signal, the function generator 110 was disconnected and the LCR meter 112 reconnected so as to continue impedance measurements up to the 45 h timepoint and comparisons made between the small IDE and large IDE. To provide a positive control for comparing the effects of electromediated cell death (apoptosis), the chemotherapeutic drug etoposide and a mechanical scratch were used. Etoposide is a chemotherapy drug used to treat cancer. It prevents cell proliferation by inhibiting a class of DNA repair enzymes called Topoisomerase II. Its use in the study was purely to act as a drug based positive control to induce the controlled form of cell death (apoptosis). Equally the mechanical scratch assay was a physical insult to the cell monolayer that would induce both uncontrolled necrotic and apoptotic death.

FIG. 4 is a graph showing results from the above described experiment. The graph shows a mean increase in impedance for both IDEs at the various frequencies. The increase was higher with the small IDE compared with the large IDE at 10, 20, 30, 40, and 50 kHz. Two-way ANOVA shows the mean increases with small IDE and large IDE were significantly different (p<0.001) at all the frequencies. The highest increase in impedance was detected at 10 kHz for both the small IDE and large IDE. The small IDE had an effective electrode area 16 times smaller than the effective electrode area of the large IDE.

The ratio of the mean increase with small IDE over large IDE had a mean value of 4.35. This suggests the relationship between mean increase in impedance detected due to MASMCs is inversely proportional to the electrode area.

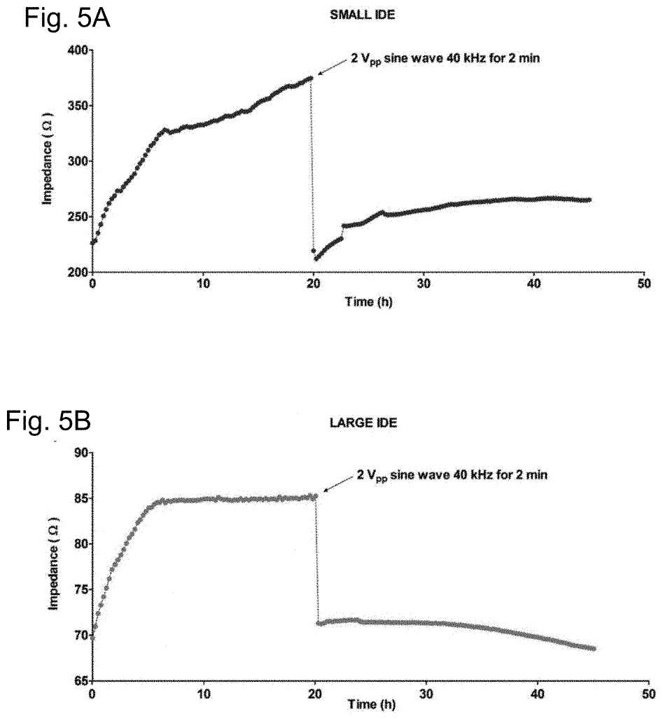

FIG. 5A and FIG. 5B are graphs that plot impedance behaviour over time. They show that both the large IDE and small IDE are able to track gradual adherence of MASMCs to the electrodes sensor.

The large IDE detected an increase of ˜15 Ω when the cells had settled down while the small IDE detected a maximum increase of ˜150 Ω. After the application of the voltage, the impedance dramatically dropped by 15Ω for the large IDE and by 160Ω for the small IDE. In both cases, the impedance dropped approximately back to the baseline impedance detected at Ωh. Following this drop, the impedance did not go back up to the pre-voltage impedance, implying that the voltage causes a permanent effect on the cells. However, any physical effect across the on the cells were not immediately observable using bright-field microscopy. Moreover, the effects seemed to be restricted to the regions in-between the electrode fingers rather than on the fingers. The cells around the IDE appeared mostly unaffected by the application of the voltage. This indicates that a very specific and localized effect can be applied to a cell monolayer.

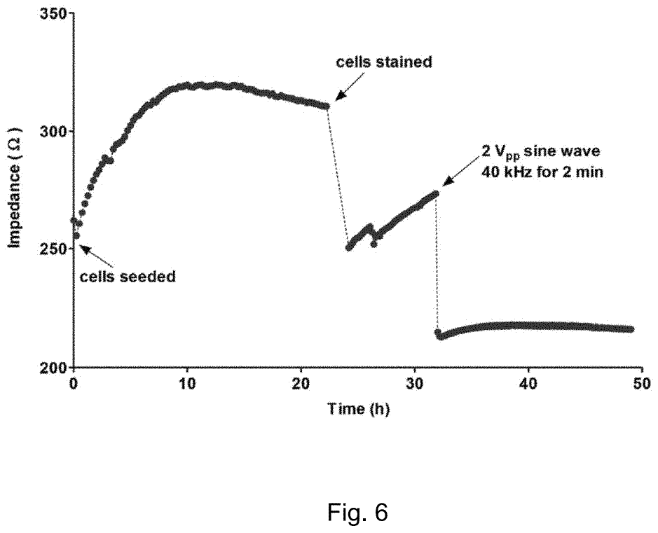

FIG. 6 is a graph showing the impedance readings for the whole duration of the experiment (49 h). The baseline impedance at 0 h was 260 0. The impedance reached a maximum of 320Ω at 10 h and gradually decreased to 310Ω at 22 h. This could be attributable to a depletion of the amount of nutrients in the media, causing the cells to detach slightly from the electrodes and establishes the current maximum duration for these types of experiments. At 22 h, the impedance measurements were stopped and the staining step was carried out. The impedance measurements were resumed at 24 h and acquired at 10 min intervals. The staining solution causes the cells to detach slightly from the electrodes, causing the impedance to drop to 250Ω. The cells were allowed to reattach to the electrodes until 32 h, where an impedance of 273Ω was reached. The voltage was applied at that point, causing an immediate drop in impedance to 215Ω. The impedance then stayed relatively constant from this point to 49 h.

Fluorescent live cell imaging was used to assess the effects across the monolayer more precisely. To mimic the effects of mechanical damage that might be expected during stent deployment to the vasculature a scratch assay was compared to a drug induced form of cell death and used as a positive control for the subsequent experiments. Etoposide (a topoisomerase inhibitor of DNA repair) was used in the drug-induced positive control.

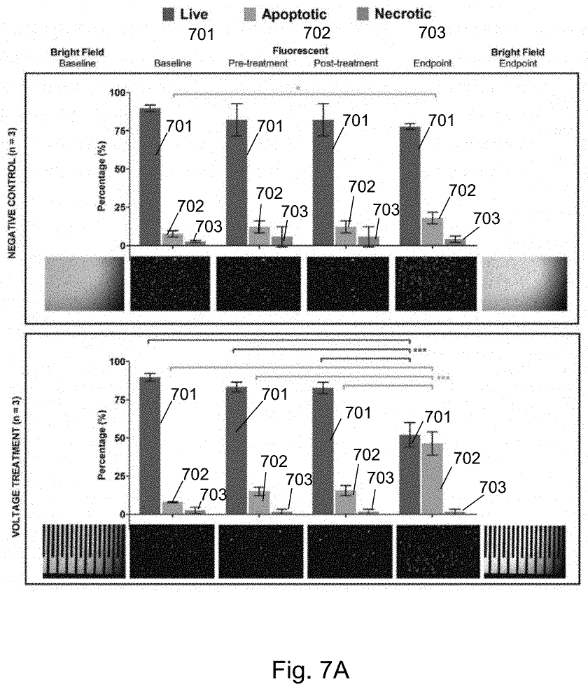

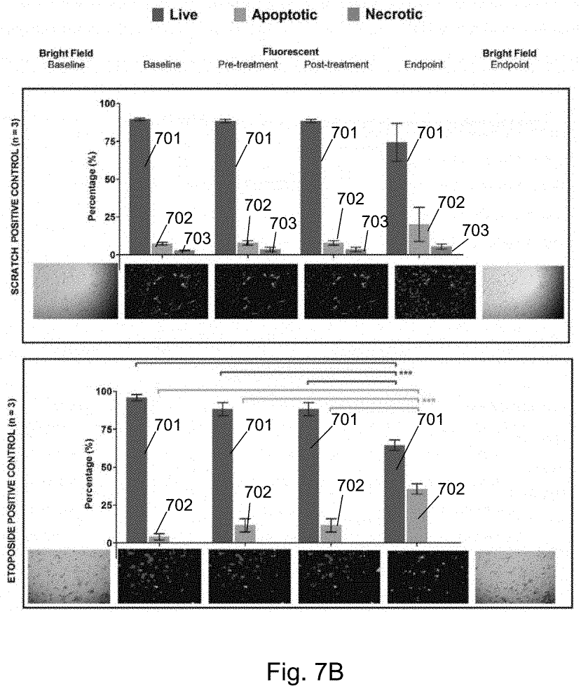

FIG. 7A is a graph showing the mean percentages of live, apoptotic and necrotic cells for the negative control and voltage treatment experiments. FIG. 7B is a graph showing the same information for the scratch positive control and the drug induced positive control. The negative control, voltage treatment and scratch positive control were carried out as part of the same experiment, while the etoposide positive control was carried out as part of a separate experiment. The live cell percentage was based on the bright field and blue fluorescence while the apoptotic and necrotic cell percentages were based on the green fluorescence and red fluorescence, respectively.

The percentages were compared using one-way ANOVA with Tukey's multiple comparison posthoc test. For the negative control, the percentage of apoptotic cells at the endpoint was statistically different (p<0.05) from the baseline, implying that there was an important increase in apoptosis from 8% to 18%. Comparing the pre-voltage treatment at baseline to the etoposide drug positive control post-treatment highlighted a statistically difference in the percentages of live and apoptotic cells by the endpoint (p<0.001 and p<0.001, respectively). This implies that the induction of apoptosis using the treatment signal had similar effects to the application of 50×10⁻⁶ m etoposide at 24 h. The percentage of apoptosis induced by the voltage was higher (46% vs 36%) compared to etoposide. Hence, a short pulse of voltage can induce a higher percentage apoptosis in a MASMC monolayer compared to a prolonged exposure to etoposide.

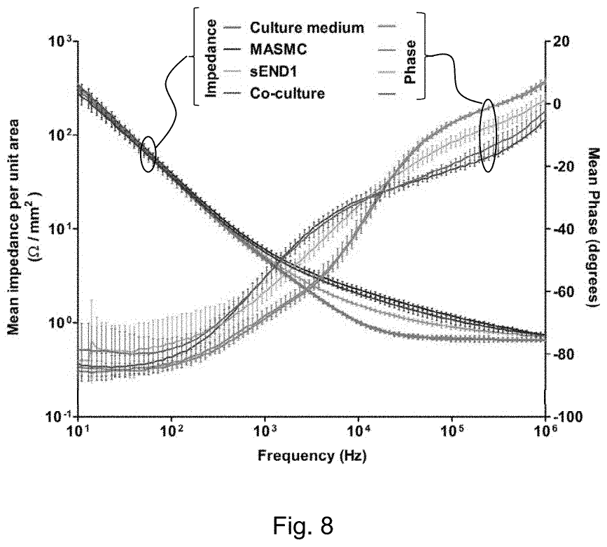

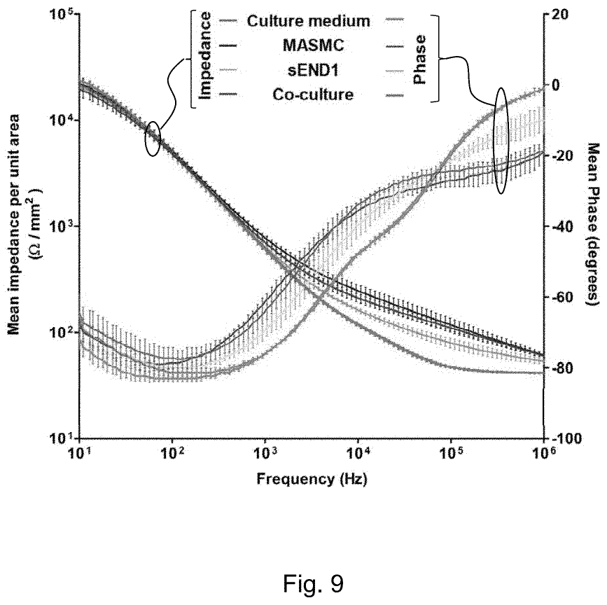

FIGS. 8 and 9 show the results of a set of Electrochemical Impedance Spectroscopy (EIS) experiments, carried out using the set up illustrated in FIG. 1, to generate impedance spectra that characterise the layers, surfaces or membranes of a sample under test. In these tests, impedance is measured while the frequency of an applied signal is swept over a large range. The results of these experiments are represented using a Bode plot, in which the magnitude of the impedance and the phase angle are plotted against the logarithm of the frequency.

In these experiments, Mouse Aortic Smooth Muscle Cells (MASMC) and immortalised murine endothelial cells (sEND1) were used. The cells were cultured in DMEM media (Sigma Aldrich, UK) supplemented with 10% Foetal Bovine Serum in culture flasks. Once sub-confluent, the cells were trypsinised and 400,000 cells were then seeded into the fabricated device, and the cell suspension was pipetted up and down a few times to ensure a homogeneous population was plated. Pure MASMC populations, pure sEND1 populations and coculture (1:1 ratio) were used in separate experiments. The large IDE and small IDE were present within the same cell chamber. Thus, the same cell monolayer covered both the large IDE and the small IDE.

The microfabricated cell chambers were sterilised by misting with 70% ethanol and rinsing with deionised water. The LCR meter was used in the constant current (CC) mode, with the current set at 10 μA. The baseline impedance spectra for the frequency range 10 Hz to 1 MHz were first measured with DMEM culture medium only. Then, 400,000 cells were seeded into the chambers and allowed to adhere to the bottom (glass+ electrodes) of the chambers for 18 h, inside an incubator (37° C. and 5% CO₂). Then the experimental impedance spectra, with the cells, were acquired.

In order to compare between the large IDE and small IDE, the impedance was normalised to impedance per unit area. As complex impedance is a vector quantity, only the magnitude of the impedance is dependent on the area of the electrode while the phase is independent of the area of the electrode. Hence, normalisation was carried out by dividing the magnitude of the impedance by the area of the respective electrodes and keeping the phase constant.

The impedance spectra were recorded using the LCR meter with 3 technical replicates (3 microfabricated chambers) for the 3 cell populations (MASMCs, sEND1 and co-culture). FIG. 8 shows a graph of mean impedance per unit area (MIPUA) vs frequency for the large IDE, and FIG. 9 shows a graph of mean impedance per unit area (MIPUA) vs frequency for the small IDE. All data in the graphs are representative of replicate data samples with a mean±standard deviation (SD).

The graphs shown in FIGS. 8 and 9 can be analysed using Area Under Curve (AUC) to performed two comparisons:
1. Between the same cell populations (MASMC, sEND1 and co-culture) and different electrodes (LIDE & SIDE).
2. Between the same electrode types and different cell populations.

For the MIPUA vs Frequency plots, the AUC increase was calculated by subtracting the AUC of the “culture medium only” curve from the AUC of the “culture medium+ cells” curve. The AUC increase was compared between different pairs of cell populations and electrode types using Oneway ANOVA.

For the large IDE (FIG. 8), the experimental phase spectra is above the baseline (culture medium only) between approximately 1 kHz and 10 kHz and below the baseline above 10 kHz. For the small IDE (FIG. 9), the experimental phase spectra is above the baseline between approximately 100 Hz and 100 kHz and below the baseline above 100 kHz. The impedance spectra diverge between approximately 1 kHz and 1 MHz for the large IDE, which indicates its optimum sensing frequency range. The impedance spectra diverge between approximately 1 kHz and above 1 MHz for the small IDE, which indicates its optimum sensing frequency range. The frequency sweep range of the LCR had a maximum of 1 MHz, and thus the upper end of the optimum frequency range for the small IDE could not be determined. However, this shows that miniaturising the IDE shifts the frequency parameters for cell sensing.

Within the optimum frequency range, the experimental impedance spectra is higher than the baseline impedance spectra, for both the large IDE and small IDE, indicating an increase in impedance due to cell adhering to the electrodes. AUC analysis shows that impedance increase detected by the small IDE, compared to large IDE, is significantly higher for all of MASMC, sEND1 and co-culture. This implies that, for the same cell monolayer, the small IDE can detect a higher increase in impedance per unit area compared to the large IDE. Thus, miniaturising the IDE increases its detection capability. At equal cell densities, the impedance increase detected by SIDE was significantly higher for MASMC compared to sEND1 and for co-culture compared to sEND1. The impedance increase detected by the large IDE was not significantly different for any of the cell populations. This indicates that the small IDE can detect differences in the impedance magnitude increase generated by equal cell densities.

Thus, EIS measured using the small IDE provides a way of characterising vascular cell monolayers.

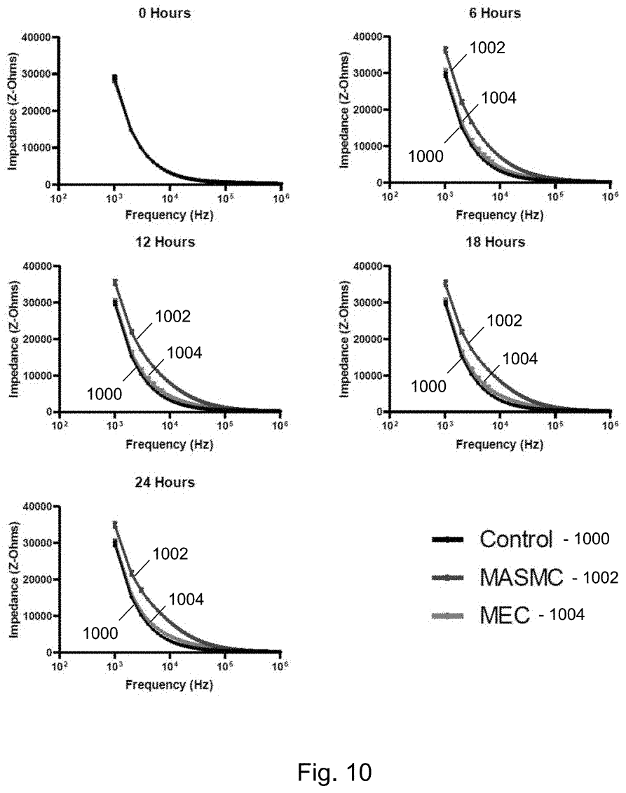

FIGS. 10-13 present results from further experiments that investigate the ability of the sensor to detect different cell types. In the context of a stent or graft, the ability to detect the difference between cell types is critical, as it will inform the physician if the interface between the stent or graft and host blood vessel is performing well and undergoing endothelial healing, or if problematic ISR is developing through smooth muscle cell hyperplasia.

In these further experiments, a setup corresponding to that shown in FIG. 1 was used. However, in these experiments the large IDE and small IDE were fabricated on a silicon substrate rather than the glass substrate used in the examples above.

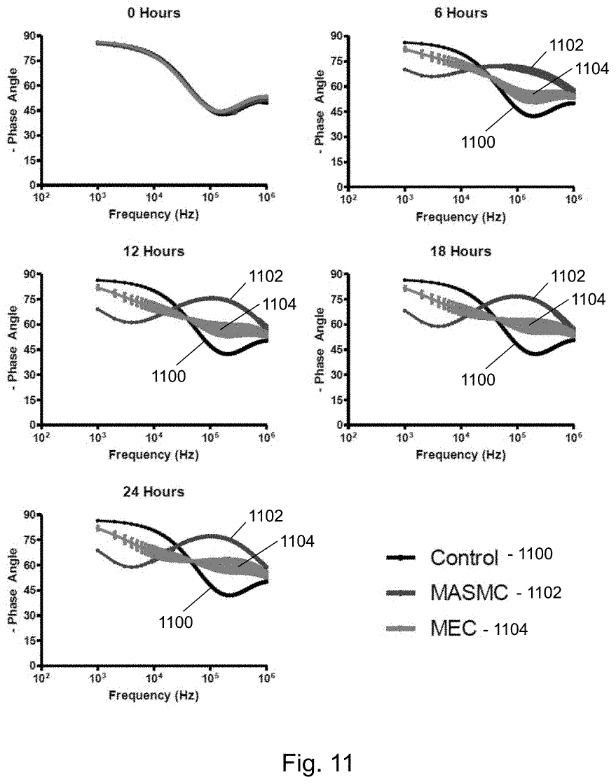

In more detail, silicon-based sensors was seeded with 50,000 mouse aorta smooth muscle cells (MASMC) or 50,000 immortalised mouse endothelial cells (MEC) or a control of media only. Impedance and phase measurements were then made from 0-24 hours at 6 hour intervals. FIG. 10 shows a set of graphs that plot impedance magnitude against frequency at each interval. FIG. 11 shows a corresponding set of graphs that plot phase against frequency.

FIG. 10 shows that, at 0hour, there was no significant difference in measured impedance between the control and the sensors seeded with cells. At 6-24 hours the impedance of sensors with

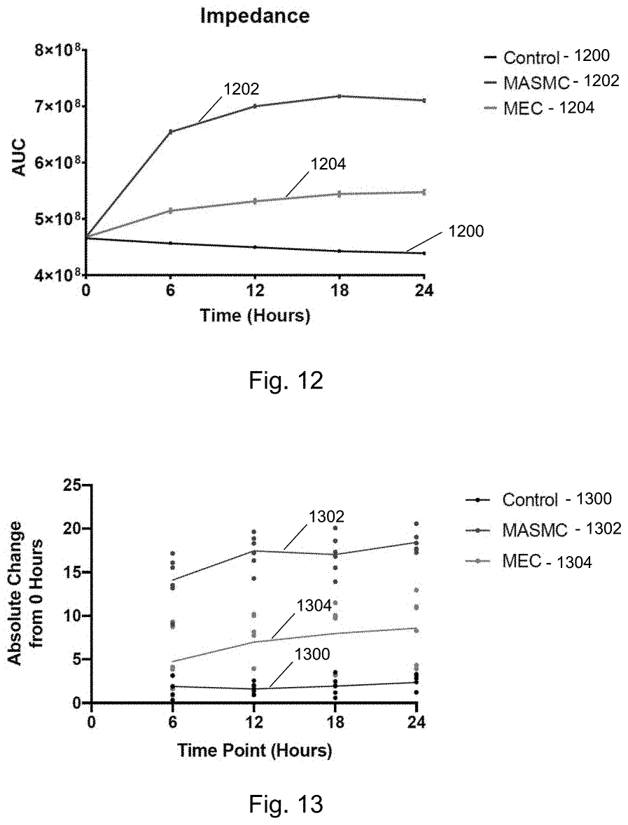

MASMCs and MECs were increased compared to the control and MASMCs had a greater impedance than MECs. Performing area under the curve (AUC) analyses for the impedances across the 1 kHz-1 MHz sweep yields the graph shown in FIG. 12. Significant differences can be seen between the control and MASMC or MEC and also between the MASMC and MEC groups. This firstly supports that different cell types seeded in identical conditions have different measured impedances. Secondly, performing AUC over the frequency sweep enables the different cell types to be distinguished.

However, it may be recognised that impedance magnitude can increase independently of cell type. For example, this may occur due to the number of cells or multiple layers of cells. Therefore, although impedance magnitude gives a basic understanding of cell type, impedance could be used more so as a way of quantifying the number of cells or even depth of cells in ISR. Thus, for reliable cell characterisation it is desirable to use a second measurement component to corroborate the impedance magnitude findings of cell type and quantity.

Using the phase angle of the impedance as a combined interpretative tool provides such corroboration and thus enables detection between phenotypically different cells. FIG. 11 shows a set of graphs that illustrate three distinct trends in phase angle over a frequency spectrum. It can be seen that over time the phase change associated with each cell type differs from the control which remains constant. For example, the profile for MASMCs has a “crescendo” rise and fall over the frequency sweep showing a decrease in phase angle between 1 kHz-10 kHz before increasing up to 100 kHz, then decreasing in the higher frequencies. Over time this trend becomes more pronounced. The profile for MECs also exhibits a change relative to the (static) control. However its overall trend is different. In contrast to the MASMCs, MECs show a linear decrease towards 10 kHz before a slight “crescendo” effect in the higher frequencies.

Similar to impedance magnitude, it is possible to analyse the phase angle over the whole frequency and compare the groups using one-way ANOVA. FIG. 13 shows a graph in which the average phase angle across the frequency spectrum for each repeat at each time point is plotted, and a line of best fit is shown for the combined average of all repeats. Comparison shows that at 0-6 hours has no difference between the control and MASMCs. Surprisingly, MECs were different to the control at 0-6 hours. The change over time for the MASMCs showed that the cells were different from the control at each time point. While MECs were not significantly different from the control at 0-6 hours but were for the remaining time points, table 2. Using average spectrum data, we can identify 3 bands or groups which correspond to the control or two type of cells. This represents a resolute but efficient, in time and complexity, foundation to determine cell types.

Additional information in cell type can be gleaned by looking at peak-to-peak differences in phase angle trends shown in FIG. 11. Filtering the phase angle for those closest to −90° the top 50 frequencies were averaged to produce a single frequency for each cell type peak. This was 26 kHz for MECs and 90 kHz for MASMCs. The phase angle at each frequency, 26 kHz and 90 kHz, for both cell types was used to calculate the difference between the phase angle at each peak. This was mean phase angle at 26 kHz minus mean phase angle at 90 kHz. At 0 hours both cell types had a large difference in phase angle peaks between the frequencies, MASMC -19.348 and MEC -19.291. At 6 hours both the MASMCs and MECs were both negative once more but the MASMCs were only marginally -0.44 compared to the MECs at -10.89. Here we observe the phase angle of the two cell types is changing differently between the two peaks and the cell types can be discriminated by this change. At 12-24 hours the MASMCs have positive change in phase angle between the peaks while MECs remained negative. The way in which the phase angle changes between the peaks for each cell type is therefore another possible way of distinguishing the two cell types.

The experiment discussed above demonstrate the feasibility of using an implantable medical device (IMD) that comprises a stent or graft with an integrated impedance sensor for cell characterisation. The impedance sensor may be configured to communicate with an extravascular transceiver, e.g. in a wearable device or portable smartphone or tablet, which in the future may form part of a central node for IMDs as part of a wireless body area network. The wireless connection from the IMD allows data from the SMART stent sensor to be sent to a secure cloud server, potentially connecting through a smartphone, or by some other transceiver unit. This extravascular transceiver will be capable of two-way communication to allow for both secure data transmission but also receiving of data, such as therapeutic care plans from physicians, changes in reading parameters or simply to send a data reading on demand.

Although described above in the context of enabling the assessment of ISR in a stent (or a similar wound responses in a graft), the system may also be employed to determine cell types in a variety of other applications, as discussed briefly below:
Atherosclerosis, where fibro-fatty plaques block coronary arteries. Symptoms of chronic ischemic angina can progress to acute myocardial infarction and stoke. If the plaque ruptures or erodes it initiates an occlusive thrombotic or embolic event.
Percutaneous coronary intervention (PCI), which is a procedure in which a patient diagnosed with an arterial blockage has an expandable metal stent deployed to the coronary vasculature to restores blood flow. These inert devices themselves damage the wall of the vessel that results in a hyper-proliferative response termed restenosis that can re-occlude the vessel. Self-reporting stents that include miniaturised versions of the proposed device would be efficacious in the treatment of these conditions.
Renal dialysis, which requires long-term vascular access. Haemodialysis filters impurities in the patients' blood when their kidney's fail. A dialysis machine returns the cleaned blood but requires venous catheterisation to pass the blood back into the patient. These short-term entry sites and longer-term arteriovenous (AV) fistula or AV graft fibrosis and block from stenosis without warning and vessel patency is lost. In long term dialysis patient when this occurs the patient may succumb to their diagnosis. Detection of vascular remodelling at these sites could be determined by the proposed device.
Chemotherapy, where patients require drugs or nutrition to be administered directly into the blood stream. A central venous catheter (CVC) often termed central venous access devices or central lines may be placed for many months or years. These external lines are referred to as PICC lines may block and need reposition, removal and re-implantation and benefit from the proposed device.
Stroke, which is a neurological debilitating condition in which the blood supply to the brain is blocked. Ischaemic stroke occurs in which a deficit of oxygen is caused by a thrombosis or emboli from an atherosclerotic plaque build-up, rupture or occlusive vessel disease termed intracranial stenosis, sometime exacerbated by hypertension. Detecting blood clots and preventing cerebral plaque rupture post stent deployment we be advantageous as currently there are no reporting technologies.
Aneurysm, which is a weakening of the wall of an artery, e.g. in the brain's cerebral circulation or any high pressure artery throughout the vasculature. A weakness in the vessel wall can cause a bulge, distention, or fatal perforation. Often asymptomatic when they rupture will cause massive internal bleeding and delamination of layers of the artery such as in the aorta. The device discussed above may be incorporated as a cell and flow sensor into an endovascular repair device.
Primary hypertension, which is caused be narrowing of the renal artery (renal stenosis) and can lead to uncontrollable high blood pressure and kidney failure. Current interventions involve renal stenting, which could benefit from the incorporation of the proposed device into renal stents and grafts.
Biliary and Oesophageal stents. Biliary stents can treat biliary obstruction, which occurs when one of the ducts that carries bile from the liver to the intestines via the gallbladder becomes blocked If left untreated it can lead to serious complications including severe infection, septicaemia, and death. Oesophageal stents are routinely used to maintain patency of the throat after cancer.
Stent malposition, where a stent is not closely aligned with the luminal surface of the vasculature. The proposed device can be used to detect the impedance signature of the wall and confirm appropriate alignment.
Endovascular leak, which occurs where the implant damages the wall of the vessel leading to drop in blood flow that can promote clot formation and prove fatal. The proposed device could detect these changes in real-time and report for immediate attention.
Infection detection, where bacterial and fungal growth on an implantable medical device could be detected by monitoring impedance.
Central line canulation blockage, in which a hollow needle inserted into a vessel to provide a continuous path for blood flow and administration of drugs becomes blocked. The sensor described herein may be us to detect build-up of a potential blockage.
Catheterisation fouling, where a hollow tube inserted into an open lumen suffers from overgrowth and blockage. The sensor described herein may be used as an early detection device.
Biofilm detection, in which the sensor discussed herein can detect bacterial overgrowth that causes implantable and surface mounted devices to be fouled.
Clinical drains, where hollow tubes are placed post-surgery to relieve blockages caused by air, blood, pus or build-up of fluids. They are placed ubiquitously for a number of other pathologies associated with inadequate drainage that are prone to clogging. Examples include chest drains, cysts drains, biliary and pancreatic surgery, thyroid, neurosurgery, urinary catheters, nasogastric tubes, orthopaedic procedures and after plastic surgery.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A system for cell characterisation in biological tissue, the system comprising:
an implantable medical device, IMD, comprising a plurality of sensing elements configured to interact with biological tissue at an implant location;
an impedance sensor configured to:
apply a sensing AC signal to the plurality of sensing elements,
vary a frequency of the AC signal across a detection range; and
detect data indicative of a complex impedance of the biological tissue at the plurality of sensing elements for a plurality of frequencies in the detection range; and
an analysis device configured to:
receive the detected data from the impedance sensor; and
determine one or more cell types in the biological tissue using information indicative of variation in phase and magnitude of the complex impedance across the plurality of frequencies.

2. The system of claim 1, wherein the impedance sensor forms part of the IMD.

3. The system of claim 1, wherein the analysis device is configured to compare the detected data to stored signature data representative of one or more known cell types.

4. The system of claim 3, wherein the stored signature data includes complex impedance spectra data indicative of a variation in phase and magnitude of complex impedance across the plurality of frequencies for each known cell type.

5. The system of claim 4, wherein the complex impedance spectra data comprises information indicative of an evolution over time of complex impedance properties for each known cell type.

6. The system of claim 3, wherein the stored signature data includes a peak-to-peak difference in phase angle change for each known cell type.

7. The system of claim 1, wherein the analysis device is configured to determine one or more cell types in the biological tissue using both phase and magnitude information in the detected data.

8. The system of claim 3, wherein the one or more known cell types comprise a smooth muscle cell type and an endothelial cell type.

9. The system of claim 1 further comprising a signal generator for applying a treatment signal to the plurality of sensing elements, wherein the signal generator forms part of the IMD.

10. The system of claim 9, wherein the IMD comprises a housing that contains the impedance sensor, the signal generator, and a controller arranged to configure the system to operate in either a sensing mode or a treatment mode.

11. The system of claim 1 further comprising a communication module configured for bi-directional communication with the analysis device.

12. The system of claim 1, wherein the plurality of sensing elements comprise a pair of interdigitated comb electrodes.

13. The system of claim 1, wherein the plurality of sensing elements comprises multiple pairs of electrodes configured to contact the biological tissue at multiple regions of the implant location.

14. The system of claim 1, wherein the IMD is an intravascular device.

15. The system of claim 1, wherein the analysis device is a remote device in wireless communication with the impedance sensor.

16. A method of characterising cells in biological tissue, the method comprising:
applying, by an impedance sensor, a sensing AC signal to a plurality of sensing elements in an implantable medical device, IMD, wherein the plurality of sensing elements are configured to interact with biological tissue at an implant location,
varying a frequency of the sensing AC signal across a detection range;
detecting, by the impedance sensor, data indicative of a complex impedance of the biological tissue at the plurality of sensing elements for a plurality of frequencies in the detection range; and
determining, by an analysis device in communication with the impedance sensor, one or more cell types in the biological tissue using information indicative of variation in phase and magnitude of the complex impedance across the plurality of frequencies.

17. The method of claim 16 including comparing, by the analysis device, the detected data with stored signature data representative of one or more known cell types.

18. The method of claim 16, wherein the step of determining one or more cell types in the biological tissue uses both phase and magnitude information in the detected data.

19. The method of claim 18, wherein the one or more known cell types comprise a smooth muscle cell type and an endothelial cell type.

20. The method of claim 16 further comprising applying a treatment signal to the pair of electrodes to cause cell damage.
